(19) United States
(12) Patent Application Publication
(10) Pub. No.: US 2023/0270645 A1
(43) Pub. Date: Aug. 31, 2023

(54) COSMETIC SUNSCREEN COMPOSITION, PROCESS FOR MANUFACTURING A COSMETIC SUNSCREEN COMPOSITION AND USE OF A COSMETIC SUNSCREEN COMPOSITION

(71) Applicant: L'OREAL, Paris (FR)

(72) Inventors: Antoniela GARDOLINSKI, Rio de Janeiro (BR); Marie BOISDRON, Shanghai (CN); Tassia HANNSHIRO, Rio de Janeiro (BR)

(21) Appl. No.: 18/005,775

(22) PCT Filed: Jul. 31, 2020
(86) PCT No.: PCT/BR2020/050299

§ 371 Date: Jan. 17, 2023

(51) Int. Cl. A61K 8/44; A61K 8/29; A61K 8/37; A61K 8/34; A61K 8/06; A61K 8/36; A61K 8/25; A61K 8/46; A61Q 17/04

(52) U.S. Cl. CPC A61K 8/445; A61K 8/29; A61K 8/375; A61K 8/345; A61K 8/062; A61K 8/361; A61K 8/25; A61K 8/466; A61Q 17/04; A61K 2800/43

(57) ABSTRACT

The present disclosure is directed to a cosmetic sunscreen composition comprising (a) an UV-filter system; (b) a skin tone color system; and (c) a surfactant system. The present disclosure is also directed to the use of a cosmetic sunscreen composition and to a process of manufacturing a cosmetic sunscreen composition.

### FIELD OF THE DISCLOSURE

The present disclosure is directed to a cosmetic sunscreen composition comprising (a) an UV-filter system; (b) a skin tone color system; and (c) a surfactant system. The present disclosure is also directed to the use of a cosmetic sunscreen composition and to a process of manufacturing a cosmetic sunscreen composition.

### BACKGROUND

The photoprotection of keratinous materials, including both skin and hair, is considered of great importance in order to protect from sun-damage, sunburn, photo-aging, as well as to decrease the chances of skin cancer development caused by exposure to ultraviolet (“UV”) radiation. There are typically two types of UVA/UVB sunscreen compositions used to accomplish photoprotection, namely, inorganic UV-filters and organic UV-filters.

The degree of UV protection afforded by a sunscreen composition is directly related to the amount and type of UV-filters contained therein. The higher the amounts of UV-filters, the greater the degree of UV protection (UVA/UVB). Particularly, sunscreen compositions must provide good protection against the sun, a measure of which is the Sun Protection Factor (SPF) value, yet have satisfactory sensory perception, such as a smooth but not greasy feel upon application.

Usually, sunscreen products may be in the form of lotions, milks, creams, gels, gel creams, foams, sprays and sticks. Such products can be anhydrous or in the form of emulsions, generally containing sunscreen actives that are solubilized, emulsified, or dispersed in a vehicle, which is topically applied onto the skin. The sunscreen actives, typically through the aid of polymers and other ingredients included in the vehicle, form a thin, protective, and often water-resistant layer on the skin.

Also, most organic sunscreen filters are oil-like and/or oil-soluble materials. High levels of sunscreen filters in sunscreen products render the products less appealing for their greasy skin feel, stickiness, long drying time, and leave shiny residue on the skin after application.

Additionally, an important problem of sunscreen compositions is that due to the great amount of sunscreen filters associated with great amount of fillers to ensure the good sensoriality and pigments in the O/W emulsion, it tends to be unstable.

In general, the cosmetic sunscreen products on the market are intended to provide matt effect on the skin. In this sense, the inventors have identified a need for a cosmetic product that encompasses/provides both high sun protection and an illuminating and glowing effect.

For the illuminating effect, it is used pearl agents that are coated with interference pigments. This coating agent brings color to formula, while the internal pearl agents are responsible for the glowing effect. This synergy brings an appearance of a natural skin, mostly perceived as an illuminated skin and healthy glow.

The challenge to manufacture a cosmetic sunscreen composition with the described properties above is due to high amount of the UV filter system combined with high amount of the skin tone color system make the cosmetic sunscreen composition unstable over the time. Thus, to overcome the drawbacks of manufacturing the cosmetic sunscreen composition of the present disclosure, a specific surfactant system was developed to allow the composition to remain stable and provide the desired glow/illuminated effect to the skin, while preserving satisfactory properties of the product.

### SUMMARY

The present disclosure is directed to a cosmetic sunscreen composition comprising (a) an UV-filter system; (b) a skin tone color system; and (c) a surfactant system. The compositions are also useful for both protecting the skin from damage caused by ultraviolet radiation and providing a highlighted and glow effect to the skin. The present disclosure is also directed to the use of a cosmetic sunscreen composition and to a process of manufacturing of a cosmetic sunscreen composition.

Other features and advantages of the present disclosure will be apparent from the following more detailed description of the desirable embodiments which illustrates, by way of example, the principles of the invention.

### DETAILED DESCRIPTION

In an embodiment, the cosmetic sunscreen composition of the present disclosure comprises:
(a) an UV-filter system;
(b) a skin tone color system; and
(c) a surfactant system.

In a preferred embodiment, the UV-filter system of the cosmetic sunscreen composition of the present invention is one or more selected from phenylbenzimidazole sulfonic acid, butyl methoxydibenzoylmethane, ethylhexyl triazone, terephthalylidene dicamphor sulfonic acid, octocrylene, homosalate, methylene bis-benzotriazolyl tetramethylbutylphenol, polyglyceryl-10 laurate, bis-ethylhexyloxyphenol methoxyphenyl triazine, titanium dioxide, and mixtures thereof.

The UV-filter system in the present cosmetic sunscreen composition is present in an amount of from about 10% by weight to about 50% by weight, relative to the total weight of the composition, and in some embodiments from about 20% by weight to about 80% by weight, and in some embodiments from about 30% by weight to about 70% by weight, such as about 40% to about 60% in relation to the total weight of the composition.

In a preferred embodiment, the skin tone color system of the cosmetic sunscreen composition of the present invention is one or more selected from mica particles, synthetic fluorphlogopite, tin oxide, titanium dioxide, and mixtures thereof.

The skin tone color system is present in the cosmetic composition in an amount of from about 1% by weight to about 35% by weight, relative to the total weight of the composition, and in some embodiments from about 5% by weight to about 10% by weight, in relation to the total weight of the composition.

In a preferred embodiment, the surfactant system of the sunscreen composition of the present invention is one or more selected from stearic acid, glyceryl stearate, PEG-100 stearate, potassium cetyl phosphate, sodium methyl stearoyl taurate, poloxamer 338, and mixtures thereof.

The surfactant system is present in the cosmetic composition in an amount of from about 0.5% by weight to about 15%, relative to the total weight of composition, preferably from about 1% by weight to about 10% by weight, relative to the total weight of the composition.

In a preferred embodiment, the sunscreen composition of the present invention further comprises cosmetically acceptable ingredients selected from additional sunscreens, perfume/fragrance, preserving agents, solvents, actives, surfactants, fatty compounds, vitamins, fillers, silicones, polymers, pigments and mixtures thereof.

In a preferred embodiment, the cosmetic sunscreen composition of the present invention may present a Sun Protection Factor ranging from 30 to 80.

In various embodiments, the cosmetic sunscreen composition of the present invention may present a Sun Protection Factor of 30, 35, 40, 45, 50, 55, 60, 65, 70 and 80.

The cosmetic sunscreen composition of the invention is in the form of an oil in water (O/W) emulsion.

In a preferred embodiment, the sunscreen cosmetic composition of the present invention has a pH of 5.5 to 6.8.

The cosmetic sunscreen composition of the invention can be used as a daily product for the skin.

The cosmetic composition of the present invention presents a high level of UV-protection in order to protect the skin from the damages of the sun, easy application, good spreadability, glow and highlight effect to the skin. Additionally, the cosmetic composition of the invention has an imperceptible touch and combining high protection with smoothness to the skin.

The invention is related to a novel combination of a skin tone color system with a UV-filter system for high SPF performance, and a surfactant system with anionic and nonionic chains in an o/w emulsion that is stable over the time.

The cosmetic sunscreen composition of the present invention is stable, with desired viscosity, appearance, color, microscopy and pH range. The cosmetic sunscreen compositions are also useful for both protecting the skin from damage caused by ultraviolet radiation and providing a highlighted and glow effect to the skin.

Particularly, the stabilization of the skin tone color system and the UV-filter system is a big challenge for formulas with high SPF. The present invention aims to bring the solution for the synergy between the pearl agents coated with titanium dioxide and iron oxide presented in the skin tone color system and the oily phase containing the UV-filter system with high SPF performance.

The interface between both systems is achieved by the surfactant system comprising anionic and non-ionic chains, aiming to mix both phases on a o/w emulsion.

The superior performance of the sunscreen cosmetic composition was demonstrated by the complete characterization of the parameters for stability, such as viscosity, appearance, color, microscopy and pH.

In another preferred embodiment, the present invention is related to the use of the sunscreen composition for the manufacture of a product to be used as sunscreen daily product.

The present invention is also related to a process of manufacturing a sunscreen composition for preventing sunburns that provides for the consumer the properties described above.

Another object of the present invention is the process for manufacturing a cosmetic sunscreen composition comprising the following steps:

Step (a): prepare an oily phase by mixing the one or more sunscreen ingredient, fatty compound, and, in the main kettle, heat these components until fusion of all raw materials;

Step (b): Prepare an aqueous phase by mixing, with shear, the water, aqueous phase UV filter, Disodium EDTA, Glycerin;

Step (c): Add the aqueous phase of step (b) to the oily phase of step (a) under mixing until complete formation of the emulsion, and add powders in a temperature under 40° C., wherein fillers and pearl agents are added with agitation until total homogenization.

The interface between them are made by a surfactant system with anionic and non-ionic chains, developed to mix both phases on an o/w emulsion.

All the confirmation of performance was proved by complete characterization parameters for stability, as viscosity, appearance, color, microscopy and pH.

The present disclosure relates to a stable cosmetic sunscreen composition including stabilized pearl agents coated with titanium dioxide and iron oxide with high performance filter system on an oil in water (o/w) emulsion.

The stable cosmetic sunscreen compositions of the present invention can comprise, consist of, or consist essentially of the essential elements and limitations of the present invention described herein, as well as any of the additional or optional ingredients, components, or limitations described herein.

### TERMS

The terms “a”, “an”, and “the” are understood to encompass the plural as well as the singular.

As used herein, the expression “at least one” means one or more and thus includes individual components as well as mixtures/combinations.

As used herein, all ranges provided are meant to include every specific range within, and combination of sub ranges between, the given ranges. For example, “from about 1%, 2% or 3% to about 5%, 10% or 15%,” includes about 1% to about 5%, about 1% to about 10%, about 1% to about 15%, about 2% to about 5%, about 2% to about 10%, about 2% to about 15%, about 3% to about 5%, about 3% to about 10%, and/or about 3% to about 15%. Other than in the operating examples, or where otherwise indicated, all numbers expressing quantities of ingredients and/or reaction conditions are to be understood as being modified in all instances by the term “about”, meaning within 15% such as within 10%, such as within 5% of the indicated number. All concentrations listed are by weight percent relative to the entire composition unless specifically described otherwise.

The term “essentially free of” as used herein means having less than 3% by weight of a particular ingredient or component and “substantially free of” as used herein means having less than 2% by weight of a particular ingredient or component.

### UV-FILTER SYSTEM

In addition to the UV-filters described above, the cosmetic sunscreen composition may comprise at least one organic (ultraviolet) UV-filter. As one skilled in the art will readily understand, by UV-filter it is meant a material that attenuates ultraviolet radiation appreciably. If two or more organic UV-filters are used, they may be the same or different.

In addition, the organic UV-filter that can be used for the present invention may be active in the UV-A and/or UV-B region. The organic UV-filter may be hydrophilic and/or lipophilic.

The organic UV-filter may be solid or liquid. The terms “solid” and “liquid” mean solid and liquid, respectively, at 25° C. under 1 atm.

The organic UV-filter can be selected from the group consisting of anthranilic compounds; dibenzoylmethane compounds; cinnamic compounds; salicylic compounds; camphor compounds; benzophenone compounds; β, β-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinol; cysteine; aromatic amino acids; peptides having an aromatic amino acid residue; and mixtures thereof.

Mention may be made, as examples of the organic UV-filter(s), of those denoted below under their INCI names, and mixtures thereof. Anthranilic compounds: menthyl anthranilates, such as marketed under the trademark “Neo Heliopan MA” by Haarmann and Reimer. The dibenzoylmethane compounds: Butyl methoxydibenzoylmethane, such as marketed in particular under the trademark “Parsol 1789” by Hoffmann-La Roche; and isopropyl dibenzoylmethane. Cinnamic compounds: Ethylhexyl methoxycinnamate, such as marketed in particular under the trademark “Parsol MCX” by Hoffmann-La Roche; isopropyl methoxycinnamate; isopropoxy methoxycinnamate; isoamyl methoxycinnamate, such as marketed under the trademark “Neo Heliopan E 1000” by Haarmann and Reimer; cinoxate (2-ethoxyethyl-4-methoxy cinnamate); DEA methoxycinnamate; diisopropyl methylcinnamate; and glyceryl ethylhexanoate dimethoxycinnamate. Salicylic compounds: Homosalate (homomentyl salicylate), such as marketed under the trademark “Eusolex HMS” by Rona/EM Industries; ethylhexyl salicylate, such as marketed under the trademark “Neo Heliopan OS” by Haarmann and Reimer; glycol salicylate; butyloctyl salicylate; phenyl salicylate; dipropyleneglycol salicylate, such as marketed under the trademark “Dipsal” by Scher; and TEA salicylate, such as marketed under the trademark “Neo Heliopan TS” by Haarmann and Reimer. Camphor compounds, in particular, benzylidenecamphor derivatives: 3-benzylidene camphor, such as manufactured under the trademark “Mexoryl SD” by Chimex; 4-methylbenzylidene camphor, such as marketed under the trademark “Eusolex 6300” by Merck; benzylidene camphor sulfonic acid, such as manufactured under the trademark “Mexoryl SL” by Chimex; camphor benzalkonium methosulfate, such as manufactured under the trademark “Mexoryl SO” by Chimex; terephthalylidene dicamphor sulfonic acid, such as manufactured under the trademark “Mexoryl SX” by Chimex; and polyacrylamidomethyl benzylidene camphor, such as manufactured under the trademark “Mexoryl SW” by Chimex. Benzophenone compounds: Benzophenone-1 (2,4-dihydroxybenzophenone), such as marketed under the trademark “Uvinul 400” by BASF; benzophenone-2 (Tetrahydroxybenzophenone), such as marketed under the trademark “Uvinul D50” by BASF; Benzophenone-3 (2-hydroxy-4-methoxybenzophenone) or oxybenzone, such as marketed under the trademark “Uvinul M40” by BASF; benzophenone-4 (hydroxymethoxy benzophonene sulfonic acid), such as marketed under the trademark “Uvinul MS40” by BASF; benzophenone-5 (Sodium hydroxymethoxy benzophenone Sulfonate); benzophenone-6 (dihydroxy dimethoxy benzophenone); such as marketed under the trademark “Helisorb 11” by Norquay; benzophenone-8, such as marketed under the trademark “Spectra-Sorb UV-24” by American Cyanamid; benzophenone-9 (Disodium dihydroxy dimethoxy benzophenonedisulfonate), such as marketed under the trademark “Uvinul DS-49” by BASF; and benzophenone-12, and n-hexyl 2-(4-diethylamino-2-hydroxybenzoyl)benzoate (such as UVINUL A+ by BASF). ββ-Diphenylacrylate compounds: Octocrylene, such as marketed in particular under the trademark “Uvinul N539” by BASF; and Etocrylene, such as marketed in particular under the trademark “Uvinul N35” by BASF. Triazine compounds: Diethylhexyl butamido triazone, such as marketed under the trademark “Uvasorb HEB” by Sigma 3V; 2,4,6-tris(dineopentyl 4′-aminobenzalmalonate)-s-triazine, bis-ethylhexyloxyphenol methoxyphenyl triazine, such as marketed under the trademark «TINOSORB S» by CIBA GEIGY, and ethylhexyl triazone, such as marketed under the trademark «UVTNUL T150»by BASF. Benzotriazole compounds, in particular, phenylbenzotriazole derivatives: 2-(2H-benzotriazole-2-yl)-6-dodecyl-4-methylpheno, branched and linear; and those described in U.S. Pat. No. 5,240,975. Benzalmalonate compounds: Dineopentyl 4′-methoxybenzalmalonate, and polyorganosiloxane comprising benzalmalonate functional groups, such as polysilicone-15, such as marketed under the trademark “Parsol SLX” by Hoffmann-LaRoche. Benzimidazole compounds, in particular, phenylbenzimidazole derivatives: Phenylbenzimidazole sulfonic acid, such as marketed in particular under the trademark “Eusolex 232” by Merck, and disodium phenyl dibenzimidazole tetrasulfonate, such as marketed under the trademark “Neo Heliopan AP” by Haarmann and Reimer. Imidazoline compounds: Ethylhexyl dimethoxybenzylidene dioxoimidazoline propionate. Bis-benzoazolyl compounds: The derivatives as described in EP-669,323 and U.S. Pat. No. 2,463,264. Para-aminobenzoic acid compounds: PABA (p-aminobenzoic acid), ethyl PABA, Ethyl dihydroxypropyl PABA, pentyl dimethyl PABA, ethylhexyl dimethyl PABA, such as marketed in particular under the trademark “Escalol 507” by ISP, glyceryl PABA, and PEG-25 PABA, such as marketed under the trademark “Uvinul P25” by BASF. Methylene bis-(hydroxyphenylbenzotriazol) compounds, such as 2,2′-methylenebis[6-(2H-benzotriazol-2-yl)-4-methyl-phenol], such as marketed in the solid form under the trademark “Mixxim BB/200” by Fairmount Chemical, 2,2′-methylenebis[6-(2H-benzotriazol-2-yl)-4-(I,I,3,3-tetramethylbutyl)phenol], such as marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197,26,184, and EP-893,119, and Drometrizole trisiloxane, such as marketed under the trademark “Silatrizole” by Rhodia Chimie or- “Mexoryl XL” by L'Oréal. Benzoxazole compounds: 2,4-bis[5-I(dimethylpropyl)benzoxazol-2-yl-(4-phenyl)imino]-6-(2-ethylhexyl)imino-I,3,5-triazine, such as marketed under the trademark of Uvasorb K2A by Sigma 3V. Screening polymers and screening silicones: The silicones described in WO 93/04665. Dimers derived from a-alkylstyrene: The dimers described in DE-19855649. 4,4-Diarylbutadiene compounds: I,I-dicarboxy(2,2′-dimethylpropyl)-4,4-diphenylbutadiene.

It is in some embodiments desirable that the organic UV-filter(s) be selected from the group consisting of: butyl methoxydibenzoylmethane, ethylhexyl methoxycinnamate, homosalate, ethylhexyl salicylate, octocrylene, phenylbenzimidazole sulfonic acid, benzophenone-3, benzophenone-4, benzophenone-5, n-hexyl 2-(4-diethylamino-2-hydroxybenzoyl)benzoate, I,r-(I,4-piperazinediyl)bis[I-[2-[4-(diethylamino)-2-hydroxybenzoyl]phenyl]-methanone 4-methylbenzylidene camphor, terephthalylidene dicamphor sulfonic acid, disodium phenyl dibenzimidazole tetrasulfonate, ethylhexyl triazone, bis-ethylhexyloxyphenol methoxyphenyl triazine, diethylhexyl butamido triazone, 2,4,6-tris(dineopentyl 4′-aminobenzalmalonate)-s-triazine, 2,4,6-tris(diisobutyl 4′-aminobenzalmalonate)-s-triazine, 2,4-bis-(n-butyl 4′-aminobenzalmalonate)-6-[(3-{1 ,3 ,3 ,3-tetramethyl-1-[(trimethylsilyloxy]-disiloxanyl}propyl)amino]-s-triazine, 2,4,6-tris-(di-phenyl)-triazine, 2,4,6-tris-(ter-phenyl)-triazine, methylene bis-benzotriazolyl tetramethylbutylphenol, drometrizole trisiloxane, polysilicone-15, dineopentyl 4′-methoxybenzalmalonate, I,I-dicarboxy(2,2′-dimethylpropyl)-4,4-diphenylbutadiene, 2,4-bis[5-I (dimethylpropyl) benzoxazol-2-yl-(4-phenyl)imino]-6-(2-ethylhexy)imino-I,3,5-triazine, camphor benzylkonium methosulfate, and mixtures thereof.

In certain embodiments, the cosmetic sunscreen compositions of the present invention include an inorganic UV-filter. The inorganic UV-filter used for the present invention may be active in the UV-A and/or UV-B region; and may be hydrophilic and/or lipophilic. The inorganic UV-filter is generally insoluble in solvents, such as water, and ethanol commonly used in cosmetics.

The inorganic UV-filter can be selected from the group consisting of silicon carbide, metal oxides which may or may not be coated, and mixtures thereof. And in some embodiments, the inorganic UV-filters are selected from pigments (mean size of the primary particles: generally from about 5 nm to about 50 nm, and in some embodiments from about 10 nm to about 50 nm) formed of metal oxides, such as, for example, pigments formed of titanium oxide (amorphous or crystalline in the rutile and/or anatase form), iron oxide, zinc oxide, zirconium oxide, or cerium oxide, which are all UV photoprotective agents that are well known per se.

In some embodiments, the inorganic UV-filters are selected from titanium oxide, zinc oxide, and, in some embodiments, titanium oxide. The inorganic UV-filter may or may not be coated. With one or more coatings such as alumina, silica, aluminum hydroxide, silicones, silanes, fatty acids or salts thereof (such as sodium, potassium, zinc, iron, or aluminum salts), fatty alcohols, lecithin, amino acids, polysaccharides, proteins, alkanolamines, waxes, such as beeswax, (meth)acrylic polymers, organic UV-filters, and (per)fluoro compounds.

### SKIN TONE COLOR SYSTEM

In addition to the constituents of the cosmetic sunscreen composition described above, the present invention comprises inorganic color pigments (i.e., inorganic pigments having at least some inorganic substrate, coating or constituent). In other embodiments, the compositions of the present invention are essentially free, substantially free or devoid of inorganic color pigments.

Suitable inorganic color pigments include any of various inorganic pigments that impart tinting or color in a cosmetic composition, such as iron oxides, ultramarine blue pigments, manganese violet, ferric ferrocyanide and chromium green pigments, pearlescent pigments, effect pigments, and the like. The color pigments may be coated or uncoated, however, in certain notable embodiments uncoated color pigments are used to make the compositions. In certain embodiments, the inorganic color pigments include or consist of iron oxide.

Any of various cosmetic grades of color pigments are suitable for use in compositions of the present invention. In certain embodiments, the color pigments have an average particle size in a range from about 1 micron to about 100 microns, such as from about 1 micron to about 10 microns.

The uncoated iron oxide pigments are, for example, marketed by Arnaud under the trademarks “Nanogard WCD 2002 (FE 45B)”, “Nanogard Iron FE 45 BL AQ”, “Nanogard FE 45R AQ”, and “Nanogard WCD 2006 (FE 45R)”, or by Mitsubishi under the trademark “TY-220”.

The coated iron oxide pigments are, for example, marketed by Arnaud under the trademarks “Nanogard WCD 2008 (FE 45B FN)”, “Nanogard WCD 2009 (FE 45B 556)”, “Nanogard FE 45 BL 345”, and “Nanogard FE 45 BL”, or by BASF under the trademark “Oxyde de fer transparent”.

### SURFACTANT SYSTEM

In addition to the constituents of the surfactant system described above, the cosmetic sunscreen composition of the present invention may comprise at least one surfactant selected from the group of anionic surfactants, non-ionic surfactants and mixtures thereof.

Non-limiting examples of anionic surfactants for the embodiment of the present invention are selected from the group comprising alkyl sulfates, alkyl phosphates, alkyl ether sulfates, alkyl ether phosphates, alkylamido ether sulfates, alkylaryl polyether sulfates, monoglyceride sulfates, sulfonates, such as alkylsulfonates, alkylamide sulfonates, alkylarylsulfonates, alpha-olefin sulfonates, paraffin sulfonates, sulfosuccinates, alkylsulfosuccinates, alkyl ether sulfosuccinates, alkylamide sulfosuccinates, alkyl sulfoacetates, acylsarcosinates, acylglutamates, alkylsulfosuccinamates, taurates and N-acyl N-methyltaurates, isethionates, N-acylisethionates, N-acyltaurates, phosphates and alkyl phosphates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group. Some of these compounds may be oxyethylenated and then preferably comprise from 1 to 50 ethylene oxide units.

Non limiting examples of non-ionic surfactants for the embodiment of the present invention include, for example, alkyl- and polyalkyl- esters of glycerol, such as glyceryl stearate, polyglyceryl-3 dicitrate/stearate, mixtures of alkyl- and polyalkyl-esters of glycerol with polyglyceryl, such as polyglyceryl-3 methylglucose distearate, oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters, for instance sucrose stearate; fatty alcohol ethers of sugars, especially alkyl polyglucosides (APGs) such as decyl glucoside, lauryl glucoside, cetostearyl glucoside, optionally as a mixture with cetostearyl alcohol, and also arachidyl glucoside, for example in the form of a mixture of arachidyl alcohol, behenyl alcohol and arachidyl glucoside.

According to one particular embodiment of the invention, the mixture of the alkyl polyglucoside as defined above with the corresponding fatty alcohol may be in the form of a self-emulsifying composition. Mention may also be made of lecithins and derivatives (e.g. Biophilic), sugar esters and sodium stearoyl lactylate.

Non-limiting examples of additional surfactants suitable for use are fatty acids, glyceryl esters in addition to glyceryl stearate, alkoxylated fatty alcohols, such as stearic acid, laureth-12, glyceryl isostearate, disodium stearoyl glutamate, potassium cetyl phosphate, poloxamer 338, sodium methyl stearoyl taurate and mixtures thereof.

### NON UV-ABSORBING FATTY COMPOUNDS

In addition to the constituents of the cosmetic sunscreen composition described above, the composition of the present invention may also include additional fatty compounds selected from oils, waxes, fatty acids, fatty alcohols, and mixtures thereof. The non UV-absorbing fatty compounds do not appreciably absorb or scatter ultraviolet radiation. Accordingly, as used herein, the term “additional fatty compounds” does not include UV-filters described above described above.

Suitable non UV-absorbing fatty compounds include those suitable for providing emolliency to the skin. Examples of such compounds generally insoluble in water and includes a hydrophobic moiety, such as one meeting one or more of the following three criteria: (a) has a carbon chain of at least six carbons in which none of the six carbons is a carbonyl carbon or has a hydrophilic moiety (defined below) bonded directly to it; (b) has two or more alkyl siloxy groups; or (c) has two or more oxypropylene groups in sequence.

The hydrophobic moiety may include linear, cyclic, aromatic, saturated or unsaturated groups. The hydrophobic compound is in certain embodiments not amphiphilic and, as such, in this embodiment does not include hydrophilic moieties, such as anionic, cationic, zwitterionic, or nonionic groups, that are polar, including sulfate, sulfonate, carboxylate, phosphate, phosphonate, ammonium, including mono-, di-, and trialkylammonium species, pyridinium, imidazolinium, amidinium, poly(ethyleneiminium), ammonioalkylsulfonate, ammonioalkylcarboxylate, amphoacetate, and poly(ethyleneoxy)sulfonyl moieties. In certain embodiments, the oil does not include hydroxyl moieties.

Suitable examples of compounds of oils include vegetable oils (glyceryl esters of fatty acids, monoglycerides, diglycerides, triglycerides) and fatty esters. Specific non-limiting examples include, without limitation, esters such as isopropyl palmitate, isopropyl myristate, isononyl isonanoate, C₁₂-C₁₅ alkyl benzoates, caprylic/capric triglycerides, ethylhexyl hydroxystearate, silicone oils (such as dimethicone and cyclopentasiloxane), pentaerythritol tetraoctanoate, potassium cetyl phosphate, sodium methyl stearoyl taurate, and mineral oil. Other examples of oils include liquid organic ultraviolet filter commonly used for example as UV-absorbing sunscreens such as octocrylene, octyl salicylate, octyl methoxyxcinnamate, glyceryl stearate, PEG-100 stearate, among others.

Suitable oils include volatile and/or non-volatile oils. Such oils can be any acceptable oil including but not limited to silicone oils and/or hydrocarbon oils.

According to certain embodiments, the compositions of the present invention may include one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C.

In certain notable embodiments, the non UV-absorbing compound has a melting point of less than about 30° C. In certain other embodiments, the compositions are substantially free of waxes, e.g, non UV-absorbing fatty compounds that are solid at room temperature (about 25° C.) and atmospheric pressure (760 mm Hg, i.e., 105 Pa), which undergo a reversible solid/liquid change of state and which have a melting point of greater than 30° C., and in some embodiments, greater than about 55° C. up to about 120° C., or even as high as about 200° C.

According to certain embodiments of the invention, the cosmetic sunscreen composition according to the present invention includes an ester of sebacic acid. A notable sebacic acid ester is a diester of isopropyl alchohol and sebacic acid i.e., diisopropyl sebacate.

According to certain other embodiments the non UV-absorbing fatty compounds include diisopropyl sebacate as well as a second non UV-absorbing fatty compounds selected from diisopropyl adipate, isononyl isonanoate, and combinations thereof. In certain other embodiments, the non UV-absorbing fatty compounds include diisopropyl sebacate, diisopropyl adipate, and isononyl isonanoate.

As non-limitative examples, the preferred non UV-absorbing fatty compounds of the present invention are diisopropyl adipate, isononyl isonanoate, stearyl alcohol, and mixtures thereof.

### INORGANIC FILLER

In addition to the constituents of the cosmetic sunscreen composition described above, the cosmetic sunscreen compositions may include inorganic fillers, such silica, silica silylate, and mixtures thereof. However, in certain embodiments, compositions of the present invention are substantially free or essentially free of inorganic fillers, such as those having a median particle size from about 0.2 microns to about 1 micron.

In certain other embodiments, compositions of the present invention are substantially free or essentially free of titanium dioxide particles having a median particle size from about 0.2 microns to about 1 micron (e.g, Sachtleben Chemie of Duisburg, Germany, under the name “Hombitan FF)”.

The concentration by weight of filler in the cosmetic sunscreen composition according to the invention is generally from about 0.1% to 10% relative to the total weight of composition, preferably from about 1% by weight to about 5% by weight, relative to the total weight of the composition.

### ADDITIONAL INGREDIENTS

In addition to the essential components described hereinbefore, the composition of the invention may further comprise any usual cosmetically acceptable ingredient, which may be chosen especially from perfume/fragrance, preservative agents, antioxidants, solvents, actives, vitamins, fillers, silicones, polymers, and mixtures thereof.

A person skilled in the art will take care to select the optional additional ingredients and/or the amount thereof such that the advantageous properties of the composition according to the invention are not, or are not substantially, adversely affected by the envisaged addition.

Suitable polymers include, but are not limited to, aluminum starch octenylsuccinate, xanthan gum, poly C₁₀₋₃₀ alkyl acrylate, acrylates C₁₀₋₃₀ alkyl acrylate crosspolymer, styrene/acrylates copolymer, ammonium acryloyldimethyltaurate/vp copolymer, acrylates/C10-30 alkyl acrylate crosspolymer, and mixtures thereof, wherein the amount of polymer is from 0.1% by weight to 4% by weight, relative to the total weight of the composition.

Non-limiting example of preservative agent which can be used in accordance with the invention include phenoxyethanol, chlorphenesin, and mixtures thereof, wherein the amount of one or more solvent is from 0.1% by weight to 2% by weight, relative to the total weight of the composition. An example of antioxidant is pentaerythrityl tetra-di-t-butyl hydroxyhydrocinnamate.

Suitable solvents include, but are not limited to alcohols, glycols and polyols such as glycerin, caprylyl glycol, pentylene glycol, propylene glycol, butylene glycol, water, and mixtures thereof, wherein the amount of one or more solvent is from 0.1% by weight to 10% by weight, relative to the total weight of the composition.

Non-limiting example of vitamins suitable for the composition of the present invention includes tocopherol, wherein the amount of vitamin is from 0.1% by weight to 5% by weight, relative to the total weight of the composition.

Suitable additional actives include, but are not limited to, disodium EDTA, triethanolamine, triethanolamine, caffeine, capryloyl salicylic acid, caprylyl methicone, and mixtures thereof, wherein the amount of actives is from 0.1% by weight to 5% by weight, relative to the total weight of the composition. A non-limitative example of vegetal extract that can be included in the composition is Schisandra chinensis fruit extract.

The additional ingredients may represent from 0.5% to 35%, such as from 1% to 35% by weight of the total weight of the composition of the invention.

By way of non-limiting illustration, the invention will now be described with reference to the following examples.

### EXAMPLES

### Example I

| | | Inventive | Inventive | Inventive | Inventive |
|---|---|---|---|---|---|
| | | cosmetic | cosmetic | cosmetic | cosmetic |
| | Cosmetic | sunscreen | sunscreen | sunscreen | sunscreen |
| INCI US | type | Ex. 1 | Ex. 2 | Ex. 3 | Ex. 4 |
| Ethylhexyl triazone | Sun filter | 15-25% | 15-25% | 15-25% | 15-25% |
| Methylene bis- | | | | | |
| benzotriazolyl | | | | | |
| tetramethylbutylphenol | | | | | |
| (and) polyglyceryl-10 | | | | | |
| laurate | | | | | |
| Terephthalylidene | | | | | |
| dicamphor sulfonic acid | | | | | |
| Titanium dioxide | | | | | |
| Phenylbenzimidazole | | | | | |
| sulfonic acid | | | | | |
| Homosalate | | | | | |
| Octocrylene | | | | | |
| Bis-ethylhexyloxyphenol | | | | | |
| methoxyphenyl triazine | | | | | |
| Butyl | | | | | |
| methoxydibenzoylmethane | | | | | |
| Sodium methyl stearoyl | Surfactant | 5-10% | 5-10% | 4-8% | 4-8% |
| taurate | | | | | |
| Potassium cetyl | | | | | |
| phosphate | | | | | |
| Stearic acid | | | | | |
| Glyceryl stearate (and) | | | | | |
| peg-100 stearate | | | | | |
| Poloxamer 338 | | | | | |
| Isononyl isononanoate | Fatty | 5-10% | 5-10% | 5-10% | 5-10% |
| Diisopropyl sebacate | compound | | | | |
| Stearyl alcohol | | | | | |
| Mica (and) titanium | Pearl | 5-15% | 5-15% | 5-15% | 5-15% |
| dioxide (and) iron oxides | pigment | | | | |
| Synthetic fluorphlogopite | | | | | |
| (and) titanium dioxide | | | | | |
| (and) tin oxide | | | | | |
| Mica (and) titanium | | | | | |
| dioxide (and) iron oxides | | | | | |
| Caprylyl glycol | Solvent | 5-10% | 5-10% | 5-10% | 5-10% |
| Glycerin | | | | | |
| Pentylene glycol | | | | | |
| Disodium edta | Active | 1-5% | 1-5% | 1-5% | 1-5% |
| Caffeine | compound | | | | |
| Capryloyl salicylic acid | | | | | |
| Triethanolamine | | | | | |
| Aluminum starch | Additional | 0.1-15% | 0.1-15% | 0.1-15% | 0.1-15% |
| octenylsuccinate | ingredients | | | | |
| Acrylates/c10-30 alkyl | | | | | |
| acrylate crosspolymer | | | | | |
| Ammonium | | | | | |
| acryloyldimethyltaurate/vp | | | | | |
| copolymer | | | | | |
| Phenoxyethanol | | | | | |
| Chlorphenesin | | | | | |
| Caprylyl methicone | | | | | |
| Schisandra chinensis fruit | | | | | |
| extract | | | | | |
| Silica | | | | | |
| Silica silylate | | | | | |
| Tocopherol | | | | | |
| Fragrance | | | | | |
| Water | Water | qs | qs | qs | qs |

Stabilized formula that can perform with all parameters expected in the stability process and torture test process of the present invention, keeping the appearance, color, sensorial and spreadability expected.

### Example II

A stability test was carried out in order to demonstrate the superior performance of the cosmetic composition of Ex. 1 to Ex. 4. The formulas were submitted to vibration, wherein:
1. the formulas were set on a rigid falcon tube;
2. the formulas were submitted during 10 minutes at max vibration force; and
3. the appearance of the formulas was analyzed.

As result, it was observed that the formulas remained stable after vibration.

After that, formulas are analyzed during two months:
1 week at 55° C. (we analyze viscosity, pH and microscopy, appearance color and odor;
1 month at 45° C., room temperature and 4° C. (viscosity, pH, microscopy, appearance color and odor;

2 month at 45° C., room temperature and 4° C. (viscosity, pH, microscopy, appearance color and odor.

After this period, the formulas were analyzed and remained stable.

### Example III

A test was performed during 15 days wherein the volunteers used blind products (EX. 1 and Ex. 2).

The application frequency was the same as a usual product, but at least 5 days in the test period, and the application zone was the face of the users.

As recruitment criteria or target definition, it was selected women with 18 to 30 years-old with dry, normal and mixed skin type, wherein these women are daily users of dermo cosmetic facial sunscreen FPS50-70, tinted or not, who do not reject tinted sunscreen or illuminating/glow effect. Further, these women are of social class AB1.

As sample characteristic, it was selected a total of 80 women (40 women per shade, i.e., light shade and medium shade). The application of the product was under normal conditions of use and self-assessment by users.

To analyze the results, it was used a scale of 5 points:
Strongly agree;
Somewhat agree;
Neither agree nor disagree;
Somewhat disagree; and
Strongly disagree.

Favorable answers were presented as cumulative of answers (Strongly agree and somewhat agree) in %, wherein the results can be verified in the Table I below:

TABLE I
Claims results

| Claims | % of agreement |
|---|---|
| Hydrated skin | 100 |
| Dry touch | 94 |
| Immediate glow effect | 99 |
| Minimizes tiredness signals | 96 |
| Light cover | 99 |
| Skin visibly healthy | 99 |
| Lush skin | 91 |
| Illuminated skin | 100 |
| Skin visibly radiant | 100 |
| Sensation of rested skin throughout the day | 95 |
| Skin doesn't look oily | 95 |
| Non-oily texture | 94 |
| Enhances the shape of the face | 98 |
| Adequates to my skin tone | 98 |
| Enhances the natural lush after the use | 94 |
| Enhances the natural skin tone | 98 |
| Tan finish | 91 |
| Recover skin brightness throughout the day | 95 |
| Fresh skin sensation throughout the day | 94 |
| Doesn't leave a pulled skin sensation | 100 |
| Velvety touch | 98 |
| Soft touch | 99 |
| Less tired skin aspect at the end of the day | 96 |
| Light texture | 99 |
| Fast absorption | 99 |
| Easy to spread | 100 |
| Non-sticky | 99 |
| Doesn't accumulate in the pores | 91 |
| Natural look | 96 |

Based on the results, it is concluded that the products of Ex. 1 and Ex. 2 present all the features mentioned on Table 1, showing the beneficial and advantageous features of the cosmetic composition of the invention.

1. A cosmetic sunscreen composition, comprising:
(a) an UV-filter system;
(b) a skin tone color system, wherein the skin tone color system comprises at least one of mica particles, synthetic fluorphlogopite, tin oxide, titanium dioxide, and mixtures thereof, and wherein the skin tone color system is present in an amount of from 1% by weight to 35% by weight, relative to the total weight of the composition; and
(c) a surfactant system, wherein the surfactant system comprises at least one of stearic acid, glyceryl stearate, PEG-100 stearate, potassium cetyl phosphate, sodium methyl stearoyl taurate, poloxamer 338, and mixtures thereof, and wherein the surfactant system is present in an amount of from 0.5% by weight to 15% by weight, relative to the total weight of the composition.

2. The cosmetic sunscreen composition, according to claim 1, wherein the UV-filter system comprises one or more of anthranilic compounds; dibenzoylmethane compounds; cinnamic compounds; salicylic compounds; camphor compounds; benzophenone compounds; ββ-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinol; cysteine; aromatic amino acids; peptides having an aromatic amino acid residue; and mixtures thereof.

3. The cosmetic sunscreen composition, according to claim 1, wherein the UV-filter system is present in an amount of from 10% by weight to 50% by weight, relative to the total weight of the composition.

4. The cosmetic sunscreen composition, according to claim 1, wherein the composition further comprises one or more fatty compound selected from the group consisting of stearyl alcohol, diisopropyl sebacate, diisopropyl adipate, isononyl isonanoate, diisopropyl sebacate, diisopropyl adipate, isononyl isonanoate, diisopropyl adipate, isononyl isonanoate, and mixtures thereof.

5. The cosmetic sunscreen composition, according to claim 1, wherein the composition further comprises one or more fatty compound, which is/are from 0.1% by weight to 10% by weight, relative to the total weight of the composition.

6. The cosmetic sunscreen composition, according to claim 1, wherein the composition further comprises one or more solvent selected from glycerin, caprylyl glycol, pentylene glycol, propylene glycol, butylene glycol, water, and mixtures thereof.

7. The cosmetic sunscreen composition, according to claim 1, wherein the composition further comprises one or more solvent, which is/are present in an amount from 0.1% by weight to 10% by weight, relative to the total weight of the composition.

8. The cosmetic sunscreen composition, according to claim 1, wherein it optionally further comprises one or more of perfume/fragrance, preservative agents, antioxidants, solvents, actives, vitamins, fillers, silicones, polymers, and mixtures thereof.

9. The cosmetic sunscreen composition, according to claim 1, wherein the one or more of perfume/fragrance, preservative agents, antioxidants, solvents, actives, vitamins, fillers, silicones, polymers, and mixtures thereof, is/are present in an amount between 0.1% by weight to 15% by weight, relative to the total weight of the composition.

10. The cosmetic sunscreen composition, according to claim 1, wherein the composition is an oil-in-water (o/w) emulsion.

11. A process for manufacturing a cosmetic sunscreen composition comprising:
(a) an UV-filter system;
(b) a skin tone color system, wherein the skin tone color system comprises at least one of mica particles, synthetic fluorphlogopite, tin oxide, titanium dioxide, and mixtures thereof, and wherein the skin tone color system is present in an amount of from 1% by weight to 35% by weight, relative to the total weight of the composition; and
(c) a surfactant system, wherein the surfactant system comprises at least one of stearic acid, glyceryl stearate, PEG-100 stearate, potassium cetyl phosphate, sodium methyl stearoyl taurate, poloxamer 338, and mixtures thereof, and wherein the surfactant system is present in an amount of from 0.5% by weight to 15% by weight, relative to the total weight of the composition,
wherein the process comprises the following steps:
Step (a): prepare an oily phase by mixing the one or more sunscreen ingredient, fatty compound, and, in the main kettle, heat these components until fusion of all raw materials;
Step (b): Prepare an aqueous phase by mixing, with shear, the water, aqueous phase UV filter, Disodium EDTA, Glycerin;
Step (c): Add the aqueous phase of step (b) to the oily phase of step (a) under mixing until complete formation of the emulsion, and add powders in a temperature under 40° C., wherein fillers and pearl agents are added with agitation until total homogenization.

12. A use of a cosmetic sunscreen composition comprising:
(a) an UV-filter system;
(b) a skin tone color system, wherein the skin tone color system comprises at least one of mica particles, synthetic fluorphlogopite, tin oxide, titanium dioxide, and mixtures thereof, and wherein the skin tone color system is present in an amount of from 1% by weight to 35% by weight, relative to the total weight of the composition; and
(c) a surfactant system, wherein the surfactant system comprises at least one of stearic acid, glyceryl stearate, PEG-100 stearate, potassium cetyl phosphate, sodium methyl stearoyl taurate, poloxamer 338, and mixtures thereof, and wherein the surfactant system is present in an amount of from 0.5% by weight to 15% by weight, relative to the total weight of the composition;
wherein it is for protecting the skin from damage caused by ultraviolet radiation and providing a highlighted effect to the skin.
